(19) United States
(12) Patent Application Publication
(10) Pub. No.: US 2021/0368801 A1
(43) Pub. Date: Dec. 2, 2021

(54) COMPOSITION FOR PREPARING HYPOCHLOROUS ACID WATER, AND TEST PAPER FOR DETERMINING HYPOCHLOROUS ACID WATER

(71) Applicant: Wabian Co., Ltd., Tokyo (JP)

(72) Inventors: Masami KUBOTA, Tokyo (JP); Takao KUBOTA, Tokyo (JP); Aoi MIYAZAKI, Tokyo (JP); Yoshiyuki MIYATA, Tokyo (JP)

(21) Appl. No.: 17/329,906

(22) Filed: May 25, 2021

(30) Foreign Application Priority Data
May 27, 2020 (JP) 2020-108243
Aug. 7, 2020 (JP) 2020-143941
Dec. 10, 2020 (JP) 2020-205074

(51) Int. Cl. A01N 59/00; G01N 21/78; G01N 33/18; A01N 25/32; C01B 11/04

(52) U.S. Cl. CPC A01N 59/00; G01N 21/78; C01B 11/04; A01N 25/32; G01N 33/182

(57) ABSTRACT

A composition, which can prepare hypochlorous acid water having a pH of 5 to 7.5, a concentration of hypochlorous acid of 50 to 500 ppm, a concentration of free chlorine molecules lower than an effective chlorine concentration, the concentration of free chlorine molecules of 20 ppm or less, and a nonionized hypochlorous acid as an active ingredient when diluted with an aqueous vehicle, the composition comprising: 1) one or more selected from trichloroisocyanuric acid, dichloroisocyanuric acid and salts thereof; and 2) a buffer salt (provided that, a chlorite of an alkali metal or alkaline earth metal is included in neither the aqueous medium nor the composition).

### BACKGROUND

### Technical Field

The present invention relates to hypochlorous acid water and a composition for viral deactivation, and more particularly to a composition for viral deactivation having excellent storage stability. The present invention relates to a test paper for accurately determining effect and toxicity of hypochlorous acid which is a composition for deactivation of pathogens, and more particularly to an effective and safe hypochlorous acid composition demonstrated by the test paper.

### Related Art

Deactivation of pathogens such as viruses is an important technique in terms of prognostic and preventive measures of infections caused by pathogens, and has been performed with ethanol for disinfection, formalin fumigation, general bactericides containing hypochlorite as an effective ingredient, and the like. However, a method for safely sterilizing an entire room has not been known so far, and a method for spraying electrolytic hypochlorous acid is slightly known. However, it is also known that nonionized hypochlorous acid is generated by electrolysis of a sodium chloride aqueous solution, but nonionized hypochlorous acid water generated in this manner is only temporarily present, and is quickly decomposed into chlorine and water. For this reason, toxicity development due to chlorine has been a problem.

In addition, a tablet using trichloroisocyanuric acid in which non-electrolytic nonionized hypochlorous acid which exhibits little toxicity of hypochlorous acid or a salt thereof and is excellent in a bactericidal action is generated at the time of use, and a method of dissolving a liquid generated by dissolving the tablet in water into a dry mist and using the dry mist for sterilizing a wide area have been developed (see, for example, JP 2019-154884 A and JP 2019-156784 A). Further, application of trichloroisocyanuric acid to a bactericide is already known (see, for example, JP 2019-089781 A, JP 2019-076821 A, JP 2019-004413 A, and JP 2018-162232 A). Furthermore, it is also known that isocyanuric chloride acids typified by dichloroisocyanuric acid have deodorizing and bactericidal action (see JP 2000-247806 A, JP 2001-300545 A, JP 09-208107 A, JP 10-168425 A, and JP 2002-172155 A).

It has been found that viruses of the family Coronaviridae, typified by a novel coronavirus, cause aerosol infection and survive on a surface such as a wall surface for a long time, and an effective means for removing such pathogenic viruses that are highly resident in a living space has been required after outbreak of the novel coronavirus infection. In particular, such a material is strongly desired in space deactivation requiring safety.

In addition, it is known that nonionized hypochlorous acid is generated by electrolysis of an aqueous sodium chloride solution, but it is also known that nonionized hypochlorous acid water generated in this manner is only temporarily present and is immediately decomposed into chlorine and water. For this reason, toxicity development due to chlorine has been a problem. Nevertheless, according to an announcement of NITE, 35 ppm chlorine is considered to be effective for suppression of the coronavirus.

### SUMMARY

The present invention has been made under such circumstances, and it is an object of the present invention to provide an effective and safe means for deactivation pathogenic viruses that are highly resident in a living space. It is also an object of the present invention to provide a means for determining such effective and safe means for deactivation.

In view of such a situation, as a result of intensive research efforts to obtain an effective means for removing pathogenic viruses that are highly resident in a living space, the present inventors have found that nonionized hypochlorous acid generated by dissolving one or more selected from trichloroisocyanuric acid, dichloroisocyanuric acid and salts thereof in an aqueous carrier in the presence of a buffer salt can effectively deactivate viruses in the presence of humans.

The reason why such a nonionized hypochlorous acid exhibits an effective sterilization effect without exhibiting toxicity is that the sterilization effect exists in the nonionized hypochlorous acid itself and does not depend on a chlorine molecule generated by decomposition of the nonionized hypochlorous acid. The nonionized hypochlorous acid is stabilized in a form in which a hydrogen ion is not ionized by a buffer salt, and a hydrogen atom in the hypochlorous acid exhibits a property closer to a hydroxyl group together with an oxygen atom rather than a property as an acid. The present inventors have found that such hypochlorous acid produces a small amount of chlorine molecules.

In addition, the present inventors have found that the deactivation effect on pathogens is also higher than that of sodium hypochlorite or electrolytic hypochlorous acid water containing chlorine molecules as an active ingredient, and have also found that there is a large difference between toxicity to a living body and toxicity to pathogens.

Therefore, when using hypochlorous acids, how much such nonionized hypochlorous acid is present with respect to chlorine molecules is an index of whether it can be used safely with high effect, but there is no published data that has referred to this point. Thus, there is no method for simultaneously determining the amount of nonionized hypochlorous acid and the chlorine molecular weight. As a method for measuring chlorine derived from hypochlorous acid and chlorine derived from chlorine molecules, a DPD (N,N-diethylphenylenediamine) method and a SBT method are known (see Hitoshi Akai et al., “Technical Papers of Air-Conditioning and Sanitary Engineers of Japan”, Vol. 122, 2007, 1-6). In addition, it is known that syringaldazine also exhibits a color reaction to these chlorines (see Lin Deng et. al. Environ Sci Pollut Res Int. 2018 August; 25 (23): 23227-23235). Syringaldazine reacts with both molecular chlorine and chlorine bonded to a molecule such as hypochlorous acid in a nonionized state, and has a difference in reactivity with both. First, syringaldazine develops color with respect to molecular chlorine, and then reacts with bonded chlorine to develop color.

Specifically, syringaldazine develops color with respect to molecular chlorine in about 1 to 2 seconds, and then reacts with bonded chlorine in 3 seconds or more. In other words, the molecular chlorine concentration can be known by color development within 2 seconds after contact.

In view of such a situation, as a result of intensive research efforts to obtain an effective means for removing pathogenic viruses that are highly resident in a living space, it has been found that such an object is achieved by distinguishing and using hypochlorous acid water containing nonionized hypochlorous acid as a main ingredient and having a small content of chlorine molecules.

The present invention has been completed based on the finding first found by the present inventors described above. That is, the present invention for solving the above problems is as follows.

<1> A hypochlorous acid water having a pH of 5 to 7.5 and having a concentration of free chlorine molecules lower than an effective chlorine amount.

<2> The hypochlorous acid water according to <1>, having a pH of 5 to 7.5 and a concentration of free chlorine molecules of 10 ppm or less.

<3> A hypochlorous acid water having a pH of 5 to 7.5 and showing no color indicating an upper limit of quantification within 2 seconds when brought into contact with a syringaldazine test piece.

<4> The hypochlorous acid water according to <3>, in which the upper limit of quantification is 10 ppm.

<5> The hypochlorous acid water according to any one of <1> to <4>, having a concentration of hypochlorous acid of 50 to 500 ppm.

<6> The hypochlorous acid water according to any one of <1> to <5>, including a buffer salt.

<7> The hypochlorous acid water according to any one of <1> to <6>, including hypochlorous acid generated by hydrolysis of one or more selected from trichloroisocyanuric acid, dichloroisocyanuric acid, and salts thereof in the presence of a buffer salt.

<8> The hypochlorous acid water according to any one of <1> to <7>, in which the hypochlorous acid water is produced by diluting, with an aqueous vehicle, a composition obtained by stirring and mixing 1) one or more selected from trichloroisocyanuric acid, dichloroisocyanuric acid and salts thereof and 2) a buffer salt for 5 minutes or more.

<9> The hypochlorous acid water according to any one of <1> to <8>, in which the hypochlorous acid contained is electrolytic hypochlorous acid or non-electrolytic hypochlorous acid.

<10> The hypochlorous acid water according to any one of <1> to <9>, in which the concentration of free chlorine molecules is 0.1 times or less the concentration of hypochlorous acid.

<11> A test paper for determining effect and toxicity of hypochlorous acid, the test paper independently including a test piece that measures an effective chlorine concentration, a test piece that measures pH, and a test piece that measures a free chlorine molecule concentration.

<12> The test paper according to <11>, which is used to determine whether or not a hypochlorous acid water to be determined corresponds to the hypochlorous acid water according to any one of <1> to <10>.

<13> The test paper according to <11> or <12>, in which the test piece that measures an effective chlorine concentration is a test piece for measuring an effective chlorine amount by a DPD method or a test piece for measuring an effective chlorine amount by an SMT method, and the test piece that measures a free chlorine molecular weight is a syringaldazine test piece.

<14> The test paper according to any one of <10> to <13>, in which a comparative color chart indicating that preferred ranges in the test paper are a hypochlorous acid concentration of 50 to 500 ppm, a pH of 5 to 7, and a free chlorine molecule of 0 to 10 ppm is attached.

<15> The test paper according to any one of <10> to <14>, in which a hypochlorous acid to be determined is electrolytic hypochlorous acid or non-electrolytic hypochlorous acid.

<16> The test paper according to any one of <10> to <15>, in which a hypochlorous acid to be determined is obtained by hydrolyzing dichloroisocyanuric acid or trichloroisocyanuric acid in the presence of a buffer salt.

<17> Hypochlorous acid water determined to be effective and highly safe by the test paper according to any one of <10> to <16>.

<18> The hypochlorous acid water according to <17>, which is determined to be effective and highly safe based on the fact that the pH is 5 to 7 and the concentration of free chlorine molecules is 0 to 10 ppm, by the test paper according to any one of <10> to <16>.

<19> Hypochlorous acid water which is determined to have a concentration of hypochlorous acid of 50 to 500 ppm, a pH of 5 to 7.5, and a concentration of free chlorine molecules of 10 ppm or less, by the test paper according to any one of <10> to <16>.

<20> The hypochlorous acid water according to <18> or <19>, in which the concentration of free chlorine molecules is determined by colorimetry within a contact time of 2 seconds with a syringaldazine reagent.

<21> The hypochlorous acid water according to <20>, which shows no color indicating an upper limit of quantification within 2 seconds when brought into contact with a syringaldazine test paper.

<22> The hypochlorous acid water according to any one of <1> to <10> and <17> to <21>, which is used for viral deactivation.

<23> The hypochlorous acid water according to <22>, in which the virus is an enveloped virus.

<24> The hypochlorous acid water according to <23>, in which the enveloped virus is a virus of the family Coronaviridae.

<25> The hypochlorous acid water according to any one of <1> to <10> and <17> to <24>, which is used by spraying.

<26> A composition including 1) one or more selected from trichloroisocyanuric acid, dichloroisocyanuric acid and salts thereof, and 2) a buffer salt.

<27> A composition, which can prepare the hypochlorous acid water according to any one of <1> to <10> and <17> to <25> when diluted with an aqueous vehicle.

<28> The composition according to <25>, which can prepare the hypochlorous acid water according to any one of <1> to <10> and <17> to <25> when diluted with an aqueous vehicle.

<29> The composition according to <27> or <28>, in which the dilution is dilution with an aqueous vehicle of 100 to 10000 mass times the composition.

<30> The composition according to any one of <26> to <29>, including 1) 10% by mass or more of one or more selected from trichloroisocyanuric acid, dichloroisocyanuric acid and salts thereof, and 2) 10% by mass or more of a buffer salt.

<31> A composition produced by stirring and mixing 1) one or more selected from trichloroisocyanuric acid, dichloroisocyanuric acid and salts thereof and 2) a buffer salt for 5 minutes or more.

<32> A composition, which can prepare hypochlorous acid water having a pH of 5 to 7.5, a concentration of hypochlorous acid of 50 to 500 ppm, a concentration of free chlorine molecules lower than an effective chlorine concentration, the concentration of free chlorine molecules of 20 ppm or less, and a nonionized hypochlorous acid as an active ingredient when diluted with an aqueous vehicle, the composition including:

1) one or more selected from trichloroisocyanuric acid, dichloroisocyanuric acid and salts thereof; and 2) a buffer salt (provided that, a chlorite of an alkali metal or alkaline earth metal is included in neither the aqueous vehicle nor the composition.).

<33> The composition according to <32>, having the concentration of free chlorine molecules of 10 ppm or less.

<34> The composition according to <32> or <33>, in which the concentration of free chlorine molecules is 0.1 times or less the concentration of the hypochlorous acid.

<35> The composition according to any one of <32> to <34>, in which the concentration of free chlorine molecules is determined by colorimetry within a contact time of 2 seconds with a syringaldazine reagent.

<36> The composition according to any one of <32> to <35>, in which the effective chlorine concentration is quantified by a test piece by a DPD method or an SMT method.

<37> The composition according to any one of <32> to <36>, in which the hypochlorous acid water is used for deactivation of coronavirus belonging to the family Coronaviridae.

According to the hypochlorous water and the composition of the present invention, it is possible to provide an effective means for removing pathogens such as pathogenic viruses that are safe and highly resident in a living space. Further, according to the test paper of the present invention, it is possible to provide a means for determining such an effective and safe removal means.

### BRIEF DESCRIPTION OF DRAWINGS

FIG. 1 is a diagram showing a test paper of Example 1; and

FIG. 2 is a diagram showing a color chart for test paper determination, in which appropriate numerical values are surrounded by ellipses.

### DETAILED DESCRIPTION

<Hypochlorous Acid Water>

The hypochlorous acid water of the present invention has a pH of 5 to 7.5. The pH is more preferably 6 or more, and further preferably 6.3 or more. In addition, the hypochlorous acid of the present invention has a pH of preferably 7.3 or less and more preferably 7 or less. The pH value can be measured by a pH meter or a pH test paper that is usually commercially available.

The concentration of free chlorine molecules of the hypochlorous acid of the present invention is lower than the effective chlorine concentration, specifically 20 ppm or less, preferably 10 ppm or less, more preferably 9 ppm or less, further preferably 8 ppm or less, further preferably 7 ppm or less, further preferably 6 ppm or less, further preferably 5 ppm or less, further preferably 4 ppm or less, further preferably 3 ppm or less, further preferably 2 ppm or less, and further preferably 1 ppm or less. The concentration of free chlorine molecules of the hypochlorous acid of the present invention may be 0 ppm. This is because the active ingredient of hypochlorous acid is conventionally a free chlorine molecule, but the hypochlorous acid of the present invention is a nonionized hypochlorous acid itself.

The concentration of free chlorine molecules can be measured with a syringaldazine reagent. Syringaldazine reacts with both molecular chlorine and chlorine bonded to a molecule such as hypochlorous acid in a nonionized state, and has a difference in reactivity with both. First, syringaldazine develops color with respect to molecular chlorine, and then reacts with bonded chlorine to develop color. Specifically, syringaldazine develops color with respect to molecular chlorine in about 1 to 2 seconds, and then reacts with bonded chlorine in 3 seconds or more. In other words, the molecular chlorine concentration in hypochlorous acid water can be known by color development within 2 seconds after contact.

In addition, the hypochlorous acid water of the present invention may be an embodiment that shows no color indicating an upper limit of quantification within 2 seconds when brought into contact with a syringaldazine test paper. As described above, according to the syringaldazine test paper, the molecular chlorine concentration can be known by color development within 2 seconds after contact. Then, the limit of quantification based on the color of a commercially available syringaldazine test paper (“Residual Chlorine Test Paper Aqua Check 3” manufactured by Nissan Chemical Industries, Ltd.) is 10 ppm. Therefore, when the hypochlorous acid water shows no color indicating the upper limit of quantification within 2 seconds when brought into contact with the syringaldazine test paper, the free chlorine concentration of the hypochlorous acid water can be said to be 10 ppm or less.

In a preferred embodiment of the present invention, the concentration of hypochlorous acid is preferably 10 ppm or more, more preferably 20 ppm or more, further preferably 50 ppm or more, further preferably 100 ppm or more, and further preferably 150 ppm or more.

In addition, in a preferred embodiment of the present invention, the concentration of hypochlorous acid is preferably 500 ppm or less, more preferably 200 ppm or less, further preferably 300 ppm or less, and further preferably 500 ppm or less.

The concentration of free chlorine molecules is preferably 0.1 times or less, more preferably 0.05 times or less, and more preferably 0.02 times or less the concentration of hypochlorous acid.

The hypochlorous acid contained in the hypochlorous acid water may be either an ionized hypochlorous acid or a nonionized hypochlorous acid. The preferred embodiment includes nonionized hypochlorous acid. In a more preferred embodiment, the concentration of the nonionized hypochlorous acid is in the preferred concentration range of the hypochlorous acid described above.

In a preferred embodiment of the present invention, the hypochlorous acid water of the present invention includes a buffer salt. Suitable examples of the buffer salt include isocyanuric acid produced by decomposition of dichloroisocyanuric acid and trichloroisocyanuric acid, hydrogen carbonates such as sodium hydrogen carbonate and potassium hydrogen carbonate, citrates such as citric acid and sodium citrate, phosphates such as disodium hydrogen phosphate and monosodium dihydrogen phosphate, lactates such as boric acid, lactic acid and sodium lactate, and the like. Salts of strong acid bases such as sodium sulfate, potassium sulfate and sodium chloride also stabilize a system and are thus classified as buffer salts.

The hypochlorous acid water of the present invention can be produced by diluting a composition including 1) one or more selected from trichloroisocyanuric acid, dichloroisocyanuric acid and salts thereof and 2) a buffer salt with an aqueous vehicle.

Preferred embodiment includes hypochlorous acid water produced by diluting a composition obtained by stirring and mixing the ingredients of 1) and 2) above with an aqueous vehicle for preferably 5 minutes or more, more preferably 6 minutes or more, further preferably 7 minutes or more, further preferably 8 minutes or more, and further preferably 9 minutes or more. As the aqueous vehicle, water or an aqueous solution can be exemplified, and as the aqueous solution, an acidic aqueous solution can be suitably exemplified. The stirring and mixing may be performed by any known means, and stirring and mixing by a Henschel mixer can be suitably exemplified.

In addition, details of the composition will be described later.

The hypochlorous acid water of the present invention is preferably used for viral deactivation. In particular, it is preferably used for deactivation of enveloped virus, more specifically, virus of the family Coronaviridae.

The hypochlorous acid water of the present invention is preferably used by spraying. The spraying means is not particularly limited. Spraying by pump spray or spraying in dry mist form by an ultrasonic atomizer may be used. According to the hypochlorous acid water provided in a spray form, spatial disinfection becomes possible.

<Composition>

The present invention also relates to a composition capable of preparing hypochlorous acid water.

One embodiment of the present invention is a composition including 1) one or more selected from trichloroisocyanuric acid, dichloroisocyanuric acid and salts thereof, and 2) a buffer salt.

Moreover, one embodiment of the present invention is a composition which can prepare the hypochlorous acid water of the present invention described above when diluted with an aqueous vehicle. Specifically, the present invention also relates to a composition comprising 1) one or more selected from trichloroisocyanuric acid, dichloroisocyanuric acid and salts thereof and 2) a buffer salt, in which 3) the pH when diluted with an aqueous vehicle is 5 to 7.5, and 4) the concentration of free chlorine is 10 ppm or less.

It is preferable that the composition of the present invention does not include a chlorite of an alkali metal and a chlorite of an alkaline earth metal from the viewpoint of maximizing the effect of the nonionized hypochlorous acid as an active ingredient.

As the aqueous vehicle, water or an aqueous solution can be exemplified, and as the aqueous solution, an acidic aqueous solution can be suitably exemplified. Also, the dilution rate by the aqueous vehicle can be preferably 100 mass times or more, more preferably 200 mass times or more, and further preferably 500 mass times or more. Further, the dilution rate by the aqueous vehicle can be preferably 10,000 times or less, more preferably 8000 times or less, further preferably 6000 mass times or less, further preferably 4000 mass times or less, further preferably 2000 mass times or less, and further preferably 1000 mass times or less.

It is preferable that the aqueous vehicle does not include a chlorite of an alkali metal and a chlorite of an alkaline earth metal from the viewpoint of maximizing the effect of the nonionized hypochlorous acid as an active ingredient.

One embodiment includes a composition for viral deactivation, the composition including 1) one or more selected from trichloroisocyanuric acid, dichloroisocyanuric acid and salts thereof and 2) a buffer salt, in which 3) the pH when 500 mass times of water is added is 6 to 7.5, and 4) the concentration of free chlorine is 10 ppm or less.

As described above, the gist of the present invention is to provide a composition for antiviral deactivation that is stable, easy to use, and highly safe. As a method for realizing this, it is preferable that one or more of trichloroisocyanuric acid, dichloroisocyanuric acid and salts thereof are formulated together with a buffer salt, and if necessary, dissolved in water at the time of use to generate nonionized hypochlorous acid.

The hypochlorous acid thus generated is applied to viral deactivation as a nonionized aqueous solution. The aqueous solution should have the following two requirements (i) and (ii).

(i) The pH is 5 to 7.5.

The pH is preferably 6 or more, and further preferably 6.3 or more. In addition, the pH is preferably 7.3 or less, and more preferably 7 or less.

(ii) When the concentration of free chlorine molecules is in the range of 20 ppm or less, the concentration is 10 ppm or less.

The concentration of free chlorine molecules is preferably 9 ppm or less, more preferably 8 ppm or less, further preferably 7 ppm or less, further preferably 6 ppm or less, further preferably 5 ppm or less, further preferably 4 ppm or less, further preferably 3 ppm or less, further preferably 2 ppm or less, and further preferably 1 ppm or less. In addition, the concentration of free chlorine molecules may be 0 ppm.

Further, in a relationship with the effective chlorine concentration, the concentration of free chlorine molecules is preferably 10% or less, and further preferably 5% or less based on the effective chlorine concentration.

According to the production method of the present invention described later, free chlorine can be suppressed to about 0.1 to 0.5 ppm.

Furthermore, it is preferable to satisfy the following requirement (iii).

(iii) The effective chlorine amount is 10 ppm or more.

The effective chlorine amount is preferably 20 ppm or more, further preferably 50 ppm or more, further preferably 100 ppm or more, and further preferably 150 ppm or more. Also, the effective chlorine amount is preferably 1000 ppm or less, more preferably 500 ppm or less, further preferably 200 ppm or less, further preferably 300 ppm or less, and further preferably 500 ppm or less.

By setting the composition of the present invention within such numerical ranges, a nonionized hypochlorous acid that is not ionized into hydrogen ions and hypochlorite ions can be formed, and the virus-removing effect and safety can be enhanced. A reason for bringing about such an effect is considered to be that hypochlorous acid directly acts on the virus instead of chlorine molecules.

Such a composition can also take the form of an aqueous solution, or can also be formed into a powder composition or a solid preparation composition such as a tablet, dissolved in an aqueous carrier at the time of use, and used. For example, in a case where a solid preparation is assumed, and considering a use form of being dissolved in 1 L of an aqueous carrier and used, 200 to 300 mg of trichloroisocyanuric acid or 300 to 500 mg of sodium dichloroisocyanurate is required to generate about 200 ppm of hypochlorous acid.

The content of dichloroisocyanuric acid, trichloroisocyanuric acid and salts thereof in the composition is preferably 10% by mass or more, more preferably 20% by mass or more, further preferably 30% by mass or more, further preferably 40% by mass or more, and further preferably 50% by mass or more.

In addition, the content of dichloroisocyanuric acid, trichloroisocyanuric acid and salts thereof in the composition is preferably 90% by mass or less, more preferably 80% by mass or less, further preferably 70% by mass or less, and further preferably 65% by mass or less.

The numerical value of the content is preferably a content based on the total amount of ingredients other than water included in the composition.

In order to generate such nonionized hypochlorous acid by using one or more selected from dichloroisocyanuric acid, trichloroisocyanuric acid and salts thereof, it is preferable to use 0.1 to 5 equivalents of a buffer salt with respect to these. Suitable examples of the buffer salt include isocyanuric acid produced by decomposition, hydrogen carbonates such as sodium hydrogen carbonate and potassium hydrogen carbonate, citrates such as citric acid and sodium citrate, phosphates such as disodium hydrogen phosphate and monosodium dihydrogen phosphate, lactates such as boric acid, lactic acid and sodium lactate, and the like. Salts of strong acid bases such as sodium sulfate, potassium sulfate and sodium chloride also stabilize a system and are thus classified as buffer salts. Such a ingredient is processed into a powder, a granule or a tablet by processing according to a conventional method. Any of these can be used as a solid composition of the present invention.

The content of the buffer salt in the composition is preferably 10% by mass or more, more preferably 20% by mass or more, and further preferably 30% by mass or more.

In addition, the content of the buffer salt in the composition is preferably 90% by mass or less, more preferably 80% by mass or less, further preferably 70% by mass or less, further preferably 60% by mass or less, and further preferably 50% by mass or less.

The numerical value of the content is preferably a content based on the total amount of ingredients other than water included in the composition.

The antiviral composition of the present invention can include, in addition to the above ingredients, optional ingredients usually used for formulation for the purpose of adjusting disintegrability, improving granulation, suppressing adhesion, and the like. Suitable examples of such a ingredient include polyhydric alcohols such as polyethylene glycol, surfactants such as lauromacrogol, cellulose derivatives such as hypromellose, lubricants such as magnesium stearate, and the like.

These ingredients can be processed into a solid preparation such as a granule or a tablet, a concentrated liquid preparation to be used after dilution or the like by processing according to a conventional method.

In a preferred embodiment of the present invention, a composition can be prepared by stirring and mixing a formulation ingredient containing 1) dichloroisocyanuric acid, trichloroisocyanuric acid and salts thereof and 2) a buffer salt for preferably 5 minutes or more, more preferably 6 minutes or more, further preferably 7 minutes or more, further preferably 8 minutes or more, and further preferably 9 minutes or more. Preferably, water is not added in the stirring and mixing step. In addition, the stirring and mixing may be performed by any known means, and stirring and mixing by a Henschel mixer can be suitably exemplified.

By performing stirring and mixing in the above time ranges, the concentration of free chlorine molecules generated when the composition is dissolved in an aqueous vehicle can be reduced.

Unlike hypochlorite, nonionized hypochlorous acid generated by dissolving or diluting the composition of the present invention thus formulated in an aqueous vehicle greatly reduces toxicity to a living body while maintaining sterilization, and LD50 value of the tablet itself exceeds 1 g/mouse (estimated 100 g/Kg) with respect to a mouse. It can be seen that it is much lower than 1 g/Kg (http://www.jsia.gr.jp/data/naclo.pdf) in an electrolytic type such as sodium hypochlorite. In addition, since acidity or alkalinity is neutral or slightly acidic (pH 5 to 7.5) and preferably neutral (pH 6 to 7), there is no corrosiveness due to alkali.

Such a technique will be described below. In the trichloroisocyanuric acid, dichloroisocyanuric acid, and salts thereof, the concentration of finally generated nonionized hypochlorous acid is set to preferably 100 to 500 ppm and more preferably 150 to 300 ppm with respect to a predetermined water amount. In order to make such form, if it is assumed to be dissolved in 1 L of water, 50 to 500 mg is preferable, and 100 to 300 mg is more preferable.

When the trichloroisocyanuric acid and dichloroisocyanuric acid are dissolved in water, the buffer salt has an effect of limiting the hypochlorous acid to a nonionized hypochlorous acid rather than an ionized hypochlorous acid.

<Test Paper>

The present invention also relates to a test paper for determining effect and toxicity of hypochlorous acid, the test paper independently including a test piece that measures an effective chlorine concentration, a test piece that measures pH, and a test piece that measures a free chlorine molecule concentration.

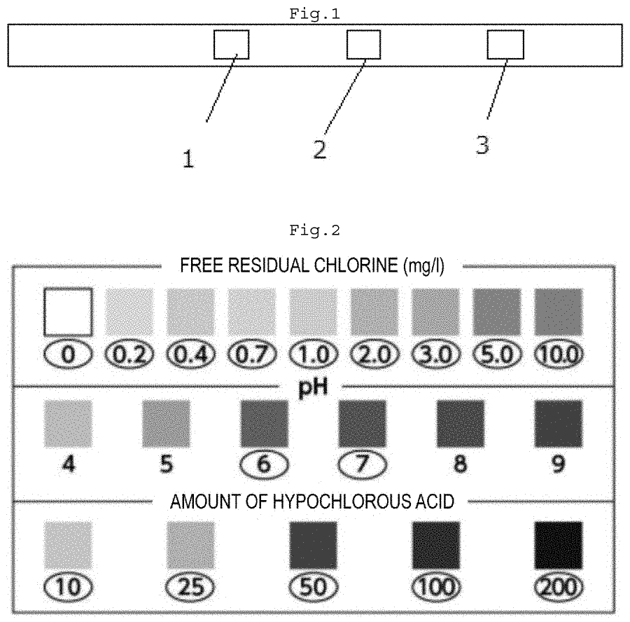

Here, the “test piece” means a test paper including at least a coloring portion and a part thereof. The scope of the present invention also includes a test paper (see FIG. 1) in which a coloring portion of a test paper that measures effective chlorine concentration, a coloring portion of a test paper that measures pH, and a coloring portion of a test paper that measures free chlorine molecule concentration are provided on the same test paper. In addition, the scope of the present invention also includes a set of test papers including the three test papers independently of each other.

As described above, the gist of the present invention is to provide a composition for antiviral deactivation that is stable, easy to use, and highly safe. Therefore, it is an object to reliably determine highly safe and highly effective hypochlorous acid, to confirm that the hypochlorous acid can be used for deactivation, and to use the hypochlorous acid for deactivation.

As a method for realizing this, it is preferable to determine the safety and the deactivation effect of non-electrolytic hypochlorous acid water obtained by formulating electrolytic hypochlorous acid water and one or more of trichloroisocyanuric acid, dichloroisocyanuric acid and salts thereof together with a buffer salt, and making it into an aqueous solution. Such hypochlorous acid water is applied to viral deactivation and pathogen deactivation.

Such highly safe and highly effective hypochlorous acid water should have the following three requirements (i) to (iii).

(i) The pH is 5 to 7.5.

The pH is preferably 6 or more, and further preferably 6.3 or more. In addition, the pH is preferably 7.3 or less, and more preferably 7 or less.

(ii) When the concentration of free chlorine molecules is in the range of 20 ppm or less, the concentration is 10 ppm or less.

The concentration of free chlorine molecules is preferably 9 ppm or less, more preferably 8 ppm or less, further preferably 7 ppm or less, further preferably 6 ppm or less, further preferably 5 ppm or less, further preferably 4 ppm or less, further preferably 3 ppm or less, further preferably 2 ppm or less, and further preferably 1 ppm or less. In addition, the concentration of free chlorine molecules may be 0 ppm.

(iii) The effective chlorine amount is 10 ppm or more.

The effective chlorine amount is preferably 20 ppm or more, further preferably 50 ppm or more, further preferably 100 ppm or more, and further preferably 150 ppm or more. Also, the effective chlorine amount is preferably 1000 ppm or less, more preferably 500 ppm or less, further preferably 200 ppm or less, further preferably 300 ppm or less, and further preferably 500 ppm or less.

It is preferable to simultaneously confirm and determine the three requirements (i) to (iii) described above. For this purpose, it is preferable that test papers coated with a substance exhibiting an accurate color reaction in these numerical ranges are combined and processed into one test paper.

(i) As a means for measuring pH, a commercially available pH test paper can be used, or a test paper prepared by dissolving a reagent such as thymol blue in alcohol or the like and spray coating the solution on paper can also be used.

(ii) As a means for measuring free chlorine concentration, it is preferable to use syringaldazine as a coloring agent, and a test paper coated with syringaldazine as a coloring reagent can also be used. The test paper can also be prepared by dissolving syringaldazine in a solvent and spraying the solution on paper.

Syringaldazine is color for measuring effective chlorine concentration at a low concentration, but rate of a color reaction with respect to hypochlorous acid-derived chlorine is different from rate of a color reaction with respect to free chlorine molecules, and the concentration of free chlorine molecules can be measured by colorimetry within 3 seconds after contact. In a commercially available test paper (“Residual Chlorine Test Paper Aqua Check 3” manufactured by Nissan Chemical Industries, Ltd.), the upper limit is 10 ppm. When the concentration exceeds this value, the color is blackened, and a change corresponding to an increase in concentration is not shown.

(iii) As a means for measuring effective chlorine amount, a reagent such as DPD or SBT is known. Products obtained by processing these into test paper are also sold and these can be used, or these reagents can also be dissolved in alcohol and coated on a paper medium.

When these test papers are commercially available products, the coloring portion is cut into small pieces of 2 to 6 mm×2 to 6 mm, and the small pieces are sequentially attached to a small piece of paper of 2 to 10 mm×30 to 100 mm with a double-sided tape or the like, whereby test paper for distinguishment can be produced. For the test paper, it is preferable to attach a reference color chart that compares the degree of color development with these numerical values (FIG. 2).

The hypochlorous acid water to be determined by the test paper of the present invention may be obtained by hydrolyzing sodium chloride, or may be obtained by hydrolyzing sodium dichloroisocyanurate with sodium hydrogen carbonate or sodium carbonate. Particularly preferred are those generated by hydrolyzing dichloroisocyanuric acid and/or an alkali metal salt thereof or trichloroisocyanuric acid having a small presence of free chlorine molecules, in other words, molecular chlorine, in the presence of a buffer salt such as a carbonate, an organic acid salt or a borate salt.

By setting hypochlorous acid water within the numerical ranges as shown in (i) to (iii) described above, a nonionized hypochlorous acid that is not ionized into hydrogen ions and hypochlorite ions can be formed, and the virus-removing effect and safety can be enhanced. A reason for bringing about such an effect is considered to be that hypochlorous acid directly acts on the virus instead of chlorine molecules.

The present invention also relates to hypochlorous acid water determined to be effective and highly safe by the test paper. Specifically, the present invention also relates to hypochlorous acid water determined to be applicable to all of the conditions (i) and (ii), and more preferably to all of the conditions (i) to (iii) by the test paper.

When such hypochlorous acid water is hydrolyzed with sodium dichloroisocyanurate and/or trichloroisocyanuric acid to prepare hypochlorous acid, 200 to 300 mg of trichloroisocyanuric acid or 300 to 500 mg of sodium dichloroisocyanurate are required in order to produce about 200 ppm of hypochlorous acid.

In order to generate such nonionized hypochlorous acid by using one or more selected from dichloroisocyanuric acid, trichloroisocyanuric acid and salts thereof, it is preferable to use 0.1 to 5 equivalents of a buffer salt with respect to these. Suitable examples of the buffer salt include isocyanuric acid produced by decomposition, hydrogen carbonates such as sodium hydrogen carbonate and potassium hydrogen carbonate, citrates such as citric acid and sodium citrate, phosphates such as disodium hydrogen phosphate and monosodium dihydrogen phosphate, lactates such as boric acid, lactic acid and sodium lactate, and the like. Salts of strong acid bases such as sodium sulfate, potassium sulfate and sodium chloride also stabilize a system and are thus classified as buffer salts. Such a ingredient is processed into a powder, a granule or a tablet by processing according to a conventional method. Any of these can be used as a solid composition of the present invention.

In one embodiment of the hypochlorous acid water determined to be effective and highly safe by the test paper of the present invention, when a color reaction is performed using the test paper, in comparison with a standard color chart, the following three requirements are satisfied: (i) the pH is 6 to 7, and more preferably 6.55 to 7; (ii) the free chlorine is 20 ppm or less and 0 to 20 ppm in the range, more preferably 10 ppm or less and 0 to 10 ppm in the range, and further preferably 8 ppm or less and 0 to 8 ppm in the range; and (iii) the effective chlorine amount is 10 to 1000 ppm and more preferably 50 to 500 ppm.

Hereinafter, the present invention will be described in more detail with reference to examples.

### Example 1

<Preparation of Test Paper>

A commercially available effective chlorine test piece of SBT method of 6 mm×6 mm, a commercially available universal test piece of 6 mm×6 mm, and a test piece prepared by dissolving 1 mg of syringaldazine in 1 ml of dimethylformamide, diluting the solution with 10 ml of methanol, uniformly spraying the resulting solution on A4 drawing paper and volatilizing the solvent to prepare test paper, and cutting paper pieces of 6 mm×6 mm from the test paper, were sequentially attached to a paper piece of 6 mm×50 mm with a double-sided tape to prepare the test paper of the present invention.

### Example 2

<Preparation of Trichloroisocyanuric Acid Tablet>

According to the following formulation, that is, after mixing the formulation ingredients with a Henschel mixer for 10 minutes, 1 g was weighed and tableted with a tableting machine to obtain a tablet. When this tablet was dissolved in 5 l of tap water, the tablet was immediately dissolved, and the solution was determined using the test paper of Example 1. As a result, the pH was 6.5, the concentration of hypochlorous acid was 100 ppm, and the free chlorine molecule concentration immediately after dissolution was 0.2 ppm. The solution had little odor.

TABLE 1

| Ingredient | % by mass |
|---|---|
| Trichloroisocyanuric acid | 52.7 |
| Sodium carbonate | 13.6 |
| Sodium hydrogen carbonate | 27.3 |
| Boric acid | 1.9 |
| Sodium sulfate | 4.5 |
| Total | 100 |

### Example 3

The same procedure as in Example 2 was carried out except that the mixing time was set to 3 minutes in the preparation of the tablet of Example 2, and the solution was measured with the test paper of Example 1. As a result, the pH was 7.5, the effective chlorine concentration was 100 ppm, and the free chlorine molecule was scaled over 10 ppm. When measured after diluting the solution to control colorimetry within the scale, the free chlorine molecule concentration was 20 ppm, and the solution had a strong irritating odor.

### Example 4

The ingredients in Table 2 were processed in the same manner as in Example 2 to obtain a composition of a powder preparation. 1.3 g of the powder preparation was weighed, and 2 L of water was added thereto to prepare an actual use solution. When the test paper of Example 1 was brought into contact with this liquid and colorimetrically compared with the standard, the pH was 6, the effective chlorine concentration was 200 ppm, and the concentration of free chlorine molecules was 10 ppm. Although the solution had some odor, it did not cause irritation.

TABLE 2

| Ingredient | % by mass |
|---|---|
| Sodium dichloroisocyanuric acid | 62.6 |
| Sodium carbonate | 10.8 |
| Sodium hydrogen carbonate | 21.5 |
| Boric acid | 1.5 |
| Sodium sulfate | 3.5 |
| Citric acid | 0.1 |
| Total | 100 |

### Example 5

A powder was prepared according to Table 3 in the same manner as in Example 2, and 1.17 g of the powder was weighed and dissolved in 2 L of water in which 0.13 mg of citric acid had been dissolved. When the test paper of Example 1 was brought into contact with this solution and colorimetrically compared with the standard color chart, the pH was 6.0, the effective chlorine concentration was 200 ppm, and the free chlorine molecule concentration was 5 ppm. The odor was very mild. Almost no irritation was felt. This shows that when sodium dichloroisocyanurate is used, it is preferable to dissolve the sodium dichloroisocyanurate in an acidic aqueous vehicle to produce an actual use solution.

TABLE 3

| Ingredient | % by mass |
|---|---|
| Sodium dichloroisocyanuric acid | 62.7 |
| Sodium carbonate | 10.8 |
| Sodium hydrogen carbonate | 21.5 |
| Boric acid | 1.5 |
| Sodium sulfate | 3.4 |
| Citric acid | 0.1 |
| Total | 100 |

### Example 6

Effect of 100 ppm actual use solution in Example 2 on porcine coronavirus was examined. Vero cells were infected with porcine coronavirus, and the virus reduction rate was calculated with and without treatment for 10 minutes with the detection limit value of TCID of the actual use solution measured in advance. 10 min after inoculation, TCID50/mL was less than 10^{0.5} (below the limit of detection), and the virus reduction rate was found to be above 99.999%. This shows that the composition of the present invention can be used for deactivation of coronavirus. In addition, since the concentration of free chlorine molecules is low, it is also found that the active body is hypochlorous acid itself. According to NITE, the effective concentration of hypochlorous acid for the coronavirus is 35 ppm as an effective chlorine amount, and this preparation far exceeds the numerical value. As described above, it can be seen that the fact that there is a difference between nonionized hypochlorous acid and normal ionizable hypochlorous acid necessitates a technique capable of accurately distinguishing the difference, and the test paper of the present invention satisfies the necessity. Therefore, hypochlorous acid water whose effect and safety have been confirmed by the test paper of the present invention has not existed as a concept at all, and the hypochlorous acid water has remarkable effect, and is completely different from conventionally known hypochlorous acid water.

### Example 7

Specimens 1 to 3 having the formulation in Table 4 were prepared on a 10 g scale (mixed by stirring 10 times for 30 seconds using a coffee mill), 0.15 g of each sample was weighed, and 500 ml of tap water was added to prepare specimens. The pH was measured with a pH meter, free chlorine molecules were colorimetrically quantified with a syringaldazine test paper, and the effective chlorine concentration was colorimetrically quantified with a test paper by a KI method.

TABLE 4

| Ingredient | Specimen 1 | Specimen 2 | Specimen 3 |
|---|---|---|---|
| Trichloroisocyanuric acid | 18 | 18 | 18 |
| Sodium sulfate | 37 | 37 | 37 |
| Sodium hydrogen carbonate | 30 | 30 | 30 |
| Sodium carbonate | 9 | 8 | 7 |
| Boric acid | 5 | 6 | 7 |
| Immediately after | | | |
| preparation | | | |
| pH | 7.8 | 7.5 | 7.1 |
| Free chlorine molecule | 10 ppm | 10 ppm | 8 ppm |
| Effective chlorine | 100 ppm | 100 ppm | 100 ppm |
| concentration | | | |
| 2 Weeks after preparation | | | |
| pH | 7.9 | 7.5 | 7.1 |
| Free chlorine molecule | 10 ppm | 10 ppm | 8 ppm |
| Effective chlorine | 100 ppm | 100 ppm | 100 ppm |
| concentration | | | |

As shown in Table 4, hypochlorous acid produced by the composition of the present invention hardly depends on pH, and is stably present even near neutrality. In addition, since the amount of free chlorine molecules hardly changes, it is inferred that these specimens are nonionized hypochlorous acid.

### Example 8

According to the formulation in Table 5, the ingredients were mixed under the condition of grinding with a mortar 5 times on a 10 g scale, and 0.3 g was weighed and dissolved in 500 ml of tap water. The pH of the solution was measured with a pH test paper, the effective chlorine concentration was measured with an effective chlorine test paper by a KI method, and the free molecular chlorine in the solution was measured with a syringaldazine test paper.

The pH was 7.5, the effective chlorine concentration exhibited a color between 50 ppm and 100 ppm, close to 50 ppm, and the free chlorine molecule under the condition of 5 times dilution with tap water was 5 ppm (that is, 25 ppm in the prepared hypochlorous acid water). In addition, the odor considered to be generation of chlorine gas was also strong.

From the above, it was confirmed that sufficient mixing was necessary in order to obtain safe hypochlorous acid water.

TABLE 5

| Ingredient | % by mass |
|---|---|
| Sodium dichloroisocyanurate | 62.6 |
| Sodium carbonate | 10.8 |
| Sodium hydrogen carbonate | 21.5 |
| Boric acid | 1.5 |
| Sodium sulfate | 3.5 |
| Citric acid | 0.1 |
| Total | 100 |

### Summary of Examples

Table 6 summarizes Examples 2 to 5. That is, the source of hypochlorous acid may be trichloroisocyanuric acid (TCCA) or dichloroisocyanuric acid or a salt thereof (DCCN), and even in the same formulation, the mass of free chlorine molecules varies depending on the production method, and thus intensity of stimulation also varies. The amount of free chlorine is preferably 20 ppm or less, and more preferably 10 ppm or less. This can be known from the color reaction of the syringaldazine of the test paper of the present invention.

TABLE 6

| | | Amount of | |
|---|---|---|---|
| | Hypochlorous | free chlorine | |
| | acid source | (ppm) | Irritating odor |
| Example 2 | Sodium | 0.2 | Little odor |
| | trichloroisocyanurate | | |
| Example 3 | Sodium | 20 | Strong |
| | trichloroisocyanurate | | irritating odor |
| Example 4 | Sodium | 10 | Some odor |
| | dichloroisocyanurate | | |
| Example 5 | Sodium | 5 | Very mild odor |
| | dichloroisocyanurate | | |

The present invention can be applied to deactivation of viruses such as coronaviruses and pathogens.

What is claimed is:

1. A composition, which can prepare hypochlorous acid water having a pH of 5 to 7.5, a concentration of hypochlorous acid of 50 to 500 ppm, a concentration of free chlorine molecules lower than an effective chlorine concentration, the concentration of free chlorine molecules of 20 ppm or less, and a nonionized hypochlorous acid as an active ingredient when diluted with an aqueous vehicle, the composition comprising:
1) one or more selected from trichloroisocyanuric acid, dichloroisocyanuric acid and salts thereof; and 2) a buffer salt (provided that, a chlorite of an alkali metal or alkaline earth metal is included in neither the aqueous vehicle nor the composition).

2. The composition according to claim 1, wherein the concentration of free chlorine molecules of 10 ppm or less.

3. The composition according to claim 1, wherein the concentration of free chlorine molecules is 0.1 times or less the concentration of the hypochlorous acid.

4. The composition according to claim 1, wherein the concentration of free chlorine molecules is determined by colorimetry within a contact time of 2 seconds with a syringaldazine reagent.

5. The composition according to claim 1, wherein the effective chlorine concentration is quantified with a test piece by a DPD method or an SMT method.

6. The composition according to claim 1, wherein the hypochlorous acid water is used for deactivation of coronavirus belonging to the family Coronaviridae.
